(19) United States
(12) Patent Application Publication
(10) Pub. No.: US 2024/0210426 A1
(43) Pub. Date: Jun. 27, 2024

(54) PORTABLE MULTIMODAL OPTICAL SENSING SYSTEM

(71) Applicants: The United States of America, as represented by the Secretary of Agriculture, Washington, DC (US); NATIONAL AGRICULTURAL PRODUCTS QUALITY MANAGEMENT SERVICE, GYEONGSANGBUK-DO (KR)

(72) Inventors: MOON S. KIM, ASHTON, MD (US); JIANWEI QIN, ELLICOTT CITY, MD (US); JO ANN S. VAN KESSEL, ELLICOTT CITY, MD (US); JEEHWA HONG, GIMCHEON-SI (KR); HYUNJEONG CHO, SEOUL (KR)

(21) Appl. No.: 18/087,391

(22) Filed: Dec. 22, 2022

(51) Int. Cl. G01N 35/00; C12Q 1/04; G01N 21/27; G01N 21/65

(52) U.S. Cl. CPC G01N 35/00029; C12Q 1/04; G01N 21/27; G01N 21/65; G01N 2201/0484

(57) ABSTRACT

The portable multimodal optical sensing system is an integrated system/tool for intelligent food safety inspection. The system includes a pair of lasers and corresponding spectrometers working at different wavelengths to enable an operator to obtain high-quality Raman scattering data from both low- and high-fluorescence food samples. By utilizing machine vision and motion control techniques, the system can conduct fully automated spectral data acquisition for randomly scattered samples that are deposited in Petri dishes or placed in customized well plates.

### FIELD OF THE INVENTION

The disclosed subject matter relates to a portable multimodal optical sensing system—preferably for automated and intelligent food safety inspections.

### BACKGROUND OF THE INVENTION

Food safety incidents are frequently reported in the news media and often result in food recalls and a rising number of food scares for consumers, which cause economic losses for food industries in both short and long terms. Foodborne outbreaks are one of the major sources for illness and death. The U.S. Centers for Disease Control and Prevention (CDC) estimates that each year 48 million people (roughly one in six Americans) are sickened or are adversely affected, 128,000 are hospitalized, and 3,000 die from foodborne diseases in the United States.

Foodborne pathogenic bacteria are linked to many outbreaks, and their opportunities to reach consumers are increasing as the farm-to-fork distance tends to grow in the global food supply chain. Three major bacteria, including Salmonella, E. coli, and Listeria, have caused more than 90% of U.S. multistate foodborne outbreaks, as highlighted by the recent E. coli O157:H7 outbreak in 27 states related to romaine lettuce in late 2019.

Food safety research explores methods to evaluate, control, and reduce potentially harmful substances in food, including both natural and introduced biological, chemical, and physical contaminants. In most food safety incidents, identification of the contaminant is usually the first step of the investigation, which generally relies on using suitable sensing technologies (e.g., optical, electrical, acoustical, and biological).

Optical sensing techniques are being investigated and developed for nondestructive food safety inspection based on different spectroscopy and imaging modalities in a wide electromagnetic spectrum, such as x-ray, ultraviolet, visible, near infrared, infrared, terahertz, fluorescence, and Raman. Despite significant progress, the high demand for safe and healthy food, strict regulations on food supply chains, and the great variety of existing and new sources of food contaminants require improved sensing technologies to achieve new levels of accuracy, speed, and intelligence.

Artificial intelligence (AI) is currently driving a new wave of innovation in many industries—including agriculture. Growth in data generation (e.g., images, videos, audios, and text), advances in algorithms (e.g., machine learning (ML) and deep learning (DL)), easy-to-use commercial and open-source software libraries (e.g., MATLAB, TensorFlow, and PyTorch), and increased computing power (e.g., personal computers, graphics processing units (GPUs)), field-programmable gate arrays (FPGAs), mobile devices, and cloud computing), AI technologies are evolving at a remarkable speed and are poised to revolutionize the agriculture industry.

Sensing technologies in the food and agriculture area are no exception. The food and agriculture industries are becoming one of the prime frontiers for AI-driven developments and transformations. For example, AI-based techniques are ideally suited for technologies such as intelligent computer vision for grain crop production and smart drying techniques for fresh foods. Deep neural networks may be used for plant phenotyping and foodborne pathogen classification.

AI-driven data analysis methods generally learn information directly from data—rather than relying on existing physical models or mathematical equations—which opens new avenues for analyzing the data collected from sensing systems. Models developed using ML and DL techniques can be used to classify and predict unknown samples, especially when the sensing signals are low or difficult to explain using the existing knowledge. For example, when classical linear chemometric methods (e.g., partial least squares (PLS)) do not suffice to extract useful information from weak spectral data of Raman or surface-enhanced Raman spectroscopy (SERS) measurements, implementing ML and DL techniques instead may lead to better results.

Recently combinations of the Raman and the DL techniques have been successfully used to rapidly identify pathogenic bacteria. In addition to the use of the AI capabilities for offline data analysis, the incorporation of the AI capabilities into the sensing systems (e.g., smart sensors and instruments) is also attracting growing attention and is established as a new developmental axis for novel sensing technologies. Pre-established ML or DL models can be deployed to companion software of the sensing systems or directly to the AI-specific hardware (e.g., AI accelerators using GPUs or FPGAs) for real-life detection applications with a level of accuracy that was not previously possible.

During the past decade, the inventors developed macro-scale Raman chemical imaging (RCI) technologies for food safety and quality research to remedy the lack of related commercial integrated systems. Two point-scan RCI systems were developed using 785 nm and 1064 nm point lasers, which are mainly used for measuring low- and high-fluorescence food samples, respectively. Each point-scan system uses a whiskbroom method for hyperspectral Raman image acquisition from samples carried by a two-axis positioning stage. A more efficient line-scan RCI system using a 5 W 785 nm line laser based on a scanning mirror was developed and patented to realize high-throughput Raman imaging for large food sample inspection.

A one-axis positioning stage is used to move the samples to accumulate hyperspectral data using a pushbroom method. The line-scan system was upgraded using a 30 W 785 nm line laser based on a cylindrical lens to enhance Raman scattering signals from the samples. Dispersive Raman spectrographs are used in both point- and line-scan systems, which can all be configured to backscattering RCI mode for surface inspection and spatially offset Raman spectroscopy (SORS) mode for subsurface inspection.

The inventors' macro-scale Raman technologies have found many practical food safety and quality applications. Examples of the RCI applications include detecting chemical adulterants mixed in food powders, lycopene changes from tomatoes during ripening, bacteria-infected watermelon seeds, veterinary drugs in pork, and bones in fish fillets. Examples of the SORS applications include nondestructive evaluation of internal maturity for tomatoes, detection of gelatin-encapsulated powders, and through-package inspection of butter for adulteration.

The 785 nm point-scan system was also configured to implement gradient temperature Raman spectroscopy (GTRS), which is a patented technique that applies the precise temperature gradients used in differential scanning calorimetry (DSC) to Raman spectroscopy. Commercial integrated Raman systems, such as FT-Raman spectrometers, Raman microscopes, and Raman microplate readers, are used in many research laboratories to provide solutions for well-defined applications. However, these systems are usually bulky and pricey, and neither flexible nor versatile enough to conduct spectroscopy and imaging experiments for food and agricultural products.

The need exists for compact automated sensing devices and methods for quick and routine measurement and analysis of chemical and biological content of sample materials. The system described herein comprises a new portable multimodal optical sensing system with embedded AI capabilities based on dual-band laser dispersive Raman techniques for automated and intelligent food safety inspection.

The system described herein was designed using modular hardware components (e.g., lasers, spectrometers, lights, cameras, and sample handling unit) that can be customized and optimized for a broad range of food safety applications. The current instrument configuraton offers more flexibility and versatility than the existing commercial Raman systems on the market.

### SUMMARY OF THE INVENTION

This disclosure is directed to a portable multimodal optical sensing system. The system includes a sample holder connected to an XY moving base. The sample holder holds at least one analyte sample. The system further comprises at least two separate cameras, at least two separate Raman laser excitation sources, and multiple illumination sources, including at least a UV light and a sample backlight. A portable base and housing at least partially encloses the XY moving base, the cameras, the Raman excitation sources, and the illumination sources. A computer/processor (which is preferably external to the portable base and housing) controls and communicates with the XY moving base, the cameras, the Raman excitation sources, and the illumination sources.

The system is structured so that the computer/processor directs the XY moving stage to enable at least one of the cameras to scan an analyte sample so that the computer/processor determines an analysis protocol for analyzing the analyte sample. The XY moving stage is configured to move in accordance with the analysis protocol so that the at least one analyte sample is analyzed to determine whether a bacterial or a chemical contaminant is present in each analyzed analyte sample. If a bacterial or chemical contaminant is present, the computer/processor identifies the bacterial or chemical contaminant.

### BRIEF DESCRIPTION OF THE DRAWINGS

The patent or application file associated with this disclosure contains at least one drawing executed in color. Copies of this patent or patent application publication with color drawing(s) will be provided by the Office upon request and payment of the necessary fee.

FIG. 1 is a perspective schematic view of the portable multimodal optical sensing system 30 disclosed herein.

FIG. 2 is an exploded view of the XY moving stage 52 based on a Petri dish sample holder 54.

FIG. 3 is a schematic view of the XY moving stage 70 based on a selected well plate sample holder 70. FIG. 3 shows multiple exemplary alternatively sized/configured well plate sample holders 75.

FIG. 4 is a flow chart describing an analysis process directed to an XY moving stage comprising a Petri dish-type sample holder 54, or an XY moving stage 70 comprising a selected well plate-type sample holder 70.

FIG. 5 is a screen shot of a software interface for monitoring and controlling the system 30 when used for sampling bacterial colonies grown on agar in a Petri dish 54.

FIG. 6 is a screen shot of a software interface for monitoring and controlling the system when used for sampling food powder samples placed in a selected well plate 75.

FIG. 7 is a flowchart showing an implementation of embedded AI functions using MATLAB and Lab VIEW for real-time target identification and labeling.

FIG. 8 shows spectral calibration for a 785 nm Raman spectrometer using polystyrene.

FIG. 9 shows spectral calibration for a 785 nm Raman spectrometer using naphthalene.

FIG. 10 shows quadratic regression of the 785 nm data shown in FIGS. 8 and 9.

FIG. 11 shows spectral calibration for a 1064 nm Raman spectrometer using polystyrene.

FIG. 12 shows spectral calibration for a 1064 nm Raman spectrometer using naphthalene.

FIG. 13 shows quadratic regression of the 1064 nm data shown in FIGS. 11 and 12.

FIG. 14 shows spatial calibration for the machine vision camera using an image acquired from a piece of 5 mm square grid paper placed in a 90 mm Petri dish.

FIG. 15 shows a backlight machine vision image for counting and locating foodborne bacterial colonies in 90 mm Petri dishes for E. coli colonies patched on a ready-to-use commercial agar plate. FIG. 15 also shows color binary images for colony counting, and colony positions for auto sampling.

FIG. 16 shows a backlight machine vision image for counting and locating foodborne bacterial colonies in 90 mm Petri dishes for Staphylococcus aureus colonies spread in a homemade agar plate. FIG. 16 also shows color binary images for colony counting, and colony positions for auto sampling.

FIG. 17 shows normalized Raman spectra for Bacillus cereus.

FIG. 18 shows normalized Raman spectra for E. coli.

FIG. 19 shows normalized Raman spectra for Listeria monocytogenes.

FIG. 20 shows normalized Raman spectra for Staphylococcus aureus.

FIG. 21 shows normalized Raman spectra for Salmonella spp.

FIG. 22 shows mean spectra of original Raman spectra for multiple bacteria, specifically: Bacillus cereus (BC); E. coli (EC); Listeria monocytogenes (LM); Staphylococcus aureus (SA); and Salmonella spp. (SS).

FIG. 23 shows mean spectra of fluorescence baselines for multiple bacteria, specifically: Bacillus cereus (BC); E. coli (EC); Listeria monocytogenes (LM); Staphylococcus aureus (SA); and Salmonella spp. (SS).

FIG. 24 shows mean spectra of corrected Raman spectra for multiple bacteria, specifically: Bacillus cereus (BC); E. coli (EC); Listeria monocytogenes (LM); Staphylococcus aureus (SA); and Salmonella spp. (SS).

FIG. 25 shows accuracies obtained from using seven classifiers on three datasets for Bacillus cereus (BC); E. coli (EC); Listeria monocytogenes (LM); Staphylococcus aureus (SA); and Salmonella spp. (SS).

FIG. 26 shows a confusion matrix for using a support vector machine (SVM) classifier with original Raman spectra for Bacillus cereus (BC); E. coli (EC); Listeria monocytogenes (LM); Staphylococcus aureus (SA); and Salmonella spp. (SS).

Note that assemblies/systems in some of the FIGs may contain multiple examples of essentially the same component. For simplicity and clarity, in some FIGs, only one (or a few) of the example components may be identified with a reference number. Unless otherwise specified, other non-referenced components with essentially the same structure as the exemplary component should be considered to be identified by the same reference number as the exemplary component. Also note that the images shown in the FIGs are not intended to be precisely to scale.

### DETAILED DESCRIPTION OF PREFERRED EMBODIMENTS

### System Capabilities

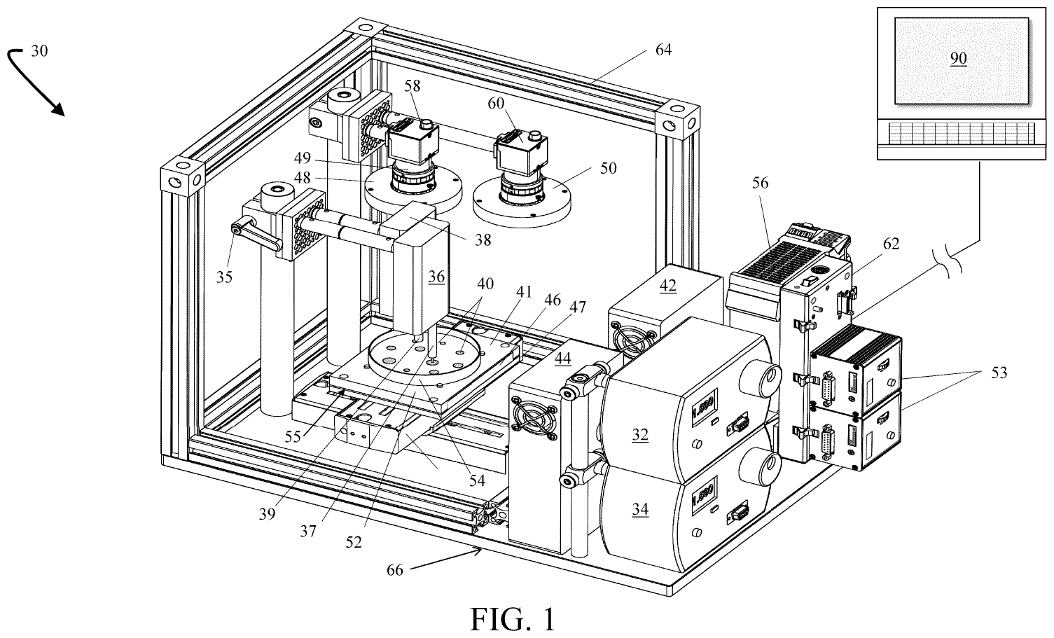

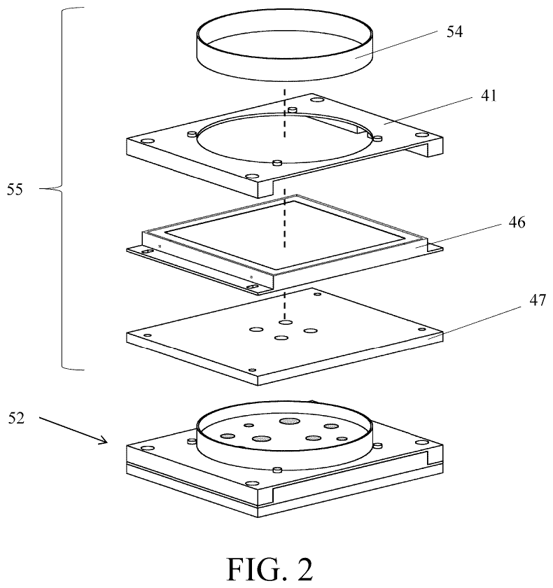

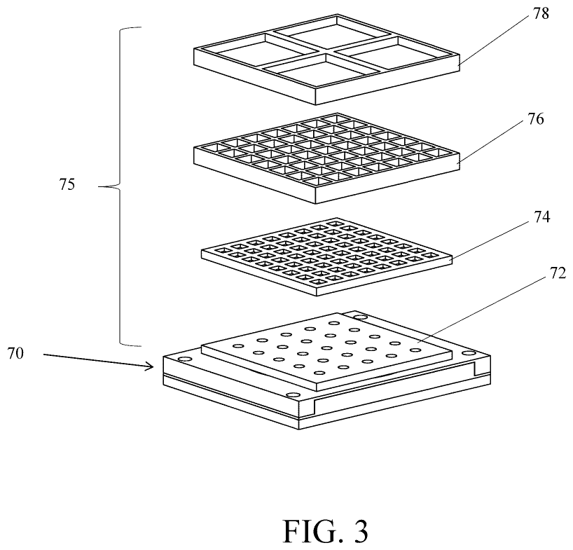

The current portable multimodal optical sensing system 30 is schematically illustrated in FIG. 1. For the purposes of this disclosure, the term “portable” means capable of being moved and carried onto a job site by a single average user/operator. The automated function of the system 30 is based on a Petri dish sample holder 54 manipulated by an XY moving stage 52 (per FIGS. 1 and 2). The system 30 alternatively comprises a well plate-based sample holder 75 that is manipulated by the XY moving stage 70 (per FIG. 3).

The system 30 is designed to analyze macro-scale samples. For the purposes of this disclosure, a “macro-scale analyte sample” means a sample with a surface area of about 5 mm or greater.

The system 30 has multiple optical sensing capabilities. The optical sensing capabilities include backlight, fluorescence, color, and Raman sensing for rapid and flexible chemical and biological sensing. The system 30 includes at least two separate color (red-green-blue known as “RGB”) image acquisition systems (i.e., “cameras”). The cameras may use active-pixel sensors such as complementary metal oxide semiconductor (CMOS) sensors. Samples are not viewed through a common camera aperture and do not depend on software-based image alignment. For the purposes of this disclosure, the term “non-common aperture” means that the system 30 includes at least two separate cameras that can be individually manually, or automatically directed to an analyte sample through two separate apertures (one aperture corresponding with each individual camera).

System data can be captured for any one or a combination of the following sensing methods: visible reflectance, backlight-based visible transmittance, excitation fluorescence, 785 nm Raman spectral imaging, 1064 nm Raman spectral imaging, and visible RGB color reflectance (photography). The system 30 includes a colony-counting function. In the automated analysis mode, the system selects the appropriate Raman laser wavelength for the analysis of each selected sample to optimize noise ratio (i.e., 785 nm and 1064 nm lasers for low- and high-fluorescence samples, respectively).

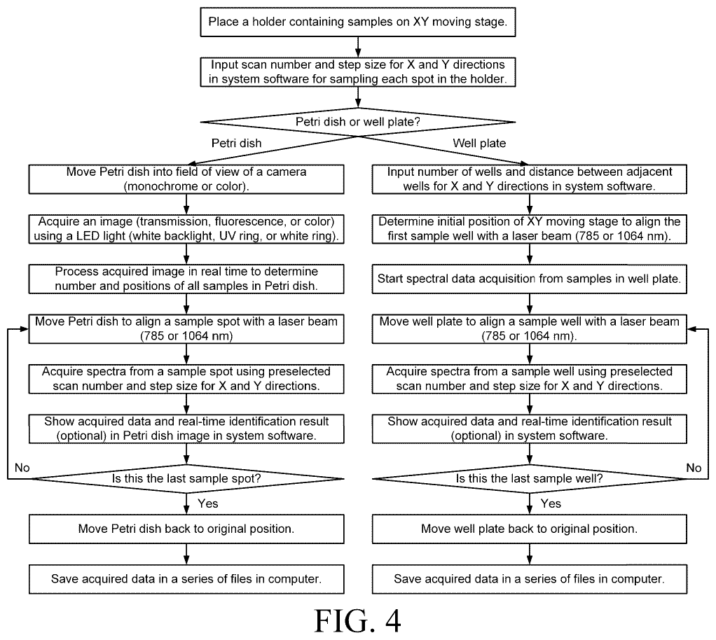

The system 30 is designed for comprehensive automated operation. Essentially, once an operator initiates the sample analysis process, the system processor/computer 90 directs at least one of the cameras to scan each of the analyte samples present in the sample holder. Additionally or alternatively, at least one of the cameras may scan one or more of the analyte samples and identify a counted number and location of targets (regions of interest) within each of the samples. Based on the types of samples present, the system computer/processor 90 determines (optionally using embedded AI) the components and an analysis protocol to examine the respective samples, which may include mechanically syncing camera imaging and/or Raman laser probe excitation and analysis. The samples are then analyzed using the general process described in the flow chart shown in FIG. 4. The system then uses AI for real-time identification of bacterial species or chemical contaminants, referencing bacteria/contaminants in a previously developed Raman spectral fingerprint library/database. For the purposes of this disclosure, the term “real-time” identification/determination means that the identification/determination result is shown essentially immediately/instantaneously (as it would be perceived by an average human user) following the acquisition and analysis of the targets in the sample.

### System Description

The system described infra is a preferred embodiment. Alternative embodiments may include variations in components, limitations, and capabilities consistent with the purpose and function of the affected system/component.

As noted supra, the system comprises at least two separate laser units 32, 34 with output wavelengths at 785 nm 32, and 1064 nm 34, (Innovative Photonic Solutions, Monmouth Junction, N.J.) which are used for Raman measurement of low- and high-fluorescence food samples, respectively. For both lasers units 32, 34, the maximum power is 450 mW with a bandwidth of 0.1 nm and a working distance of 7.2 mm.

Each laser unit 32, 34 is equipped with an adjustable laser component 36, 38 which includes a wavelength stabilized laser source with Raman filter packs and beam shaping optics. Each of the adjustable laser components 36, 38 has an associated laser probe 37, 39. The Raman probes 37, 39 are used to focus the respective lasers components 36, 38 on the sample surface 40 and collect scattering signals. Two bifurcated optical fiber bundles are used to connect two Raman laser components 36, 38 and two dispersive Raman spectrometers (QEPro for the 785 nm laser 42, and NIR Quest for the 1064 nm laser 44, Ocean Insight, Orlando, Fla.), respectively, for transferring the laser and the Raman signals. Both Raman spectrometers 42, 44 use a reflection grating to disperse the light into different wavelengths. The 785 nm spectrometer 42 uses an 18-bit back-illuminated charge-coupled device (CCD) detector with 1024 pixels to collect the dispersed light in a spectral region of 790-1020 nm. The 1064 nm spectrometer 44 uses a 16-bit InGaAs detector with 512 pixels for spectral acquisition in a wavelength range of 1070-1450 nm. An adjustable mounting clamp 35 is used to fix the two Raman laser components 36, 38 which can be vertically adjusted to accommodate different sample 40 heights.

Three LED lights, including a white backlight 46, a UV (365 nm) ring light 48, and a white ring light 50 (Advanced Illumination, Rochester, Vt.), are used to provide different illuminations for automated sampling based on machine vision measurement of the samples 40.

The underlying white backlight 46 is mounted on an XY moving stage 52, and it primarily illuminates transparent or semi-transparent samples 40 (e.g., bacterial colonies grown on agar in a Petri dish 54) for transmission image measurement. The active lighting area of the backlight 46 is 100×100 mm². The overhead UV 48 and white ring lights 50 are mainly used for fluorescence and color image measurement of non-glare samples 40, respectively. The internal and external diameters of the two identically sized ring lights 48, 50 are 35 mm and 75 mm, respectively. An LED light controller 56 with three independent outputs is used for on-off control and intensity adjustment for the three lights 46, 48, 50. Machine vision images of the samples 40 are collected by two miniature color cameras 58 and 60 with 2448×2048 pixels (Blackfly S USB3 Color, Teledyne FLIR, Wilsonville, Oreg.), each equipped with a 12 mm fixed focal length imaging lens (Edmund Optics, Barrington, N.J.). Two adapter rings are used to mount the UV 48 and white 50 ring lights on the filter thread of the lenses attached to the color cameras 58 and 60. For the color camera 58 with the UV ring light 48, a multi-band bandpass filter 49 (e.g., 538/685 nm dual-band bandpass filter or 475/543/702 nm triple-band bandpass filter, Semrock, Rochester, N.Y.) is inserted in the lens for multispectral fluorescence imaging.

A pair of direct-drive linear translation stages (DDSM100, Thorlabs, Newton, N.J.) are combined to form a two-axis moving stage 52 for positioning the samples 40 under the two Raman probes 37, 39. The movement of the stage 52 is controlled by two servo motor controllers 53 that are mounted on a USB controller hub 62, which provides power supply to the controllers 53 and USB communication to an external processor/computer 90. The XY stage 52 can move in a square area of 100×100 mm² with a maximum resolution of 0.5 μm and a maximum speed of 500 mm/s, which is ideal for high-accuracy and high-speed sample positioning for automated Raman spectral and imaging measurement. The Raman laser components 36, 38, and associated probes 37, 39, the machine vison cameras 58, 60, and lights 46, 48. 50, the XY stage 52, and the sample handling unit are housed in an aluminum-framed enclosure 64 with black foam boards (not shown) to avoid the influence of ambient light. The entire sensing system 30 is built on a 30×45 cm² optical breadboard 66, making it compact and easily portable instrument so that it is suitable for rapid field and on-site food safety inspection.

As noted supra, for the purposes of this disclosure, the term “portable” means capable of being moved and carried onto a job site by a single average operator. In the preferred embodiment, the system is about 30 cm×45 cm×35 cm and weighs about 20 kg, i.e. about 12 in ×18 in ×13.75 in and weighs about 44 pounds.

### System Operation

The system 30 can be used for both manual and automated Raman sampling. The manual measurement is conducted by manually positioning the sample 40 on a Petri dish 54 or one of the well plates (for example 76) under the Raman probes 36, 38, which is followed by XY point-scan spectral and image acquisition in a manner that is similar to that of the inventors' previously developed Raman systems. On the other hand, the system 30 can carry out fully automated sampling for samples 40 randomly scattered in Petri dishes 54 or placed in fixed patterns in customized selected well plates 75 (see FIG. 3).

As shown in FIGS. 1 and 2, in the Petri dish-based embodiments, a sample holder base 41 and a backlight base 47 are custom designed and created by a 3D printer (Fortus 250mc, Stratasys, Eden Prairie, Minn.) using production-grade thermoplastic. The two 3D printed parts 41, 47 are used to mount the white LED backlight 46 on the XY moving stage 52. The sample holder base 41 has a circular hole to hold a Petri dish 54 (e.g., 90 mm diameter) on top of the backlight 46. When analyzing a sample spot in the Petri dish 54, the XY stage 52 first moves the dish 54 into the field of view of one color camera (58 or 60), where one LED light (white backlight 46, UV ring light 48, or white ring light 50) illuminates the samples 40 in the dish and a machine vision image (transmission, fluorescence, or color) is collected by the camera 58, 60. Real-time image processing (e.g., local thresholding, morphological filtering, and particle labeling) is followed to generate a binary image to isolate the sample spots 40 from the background. Then information regarding the total number of the sample spots and the position/location of each sample spot 40 is generated and recorded. The number and location of the sample spots are used to navigate the XY stage 52 to move each spot 40 to the laser beam probe 37, 39 for continuous automated Raman measurement of all the sample spots scattered in the Petri dish 54.

For the purposes of this disclosure, a “sample spot” may also be referred to as a “region of interest” or an analyte, or, in some cases, simply a “sample”.

FIG. 2 shows an exploded view of the Petri dish sample holder/moving XY stage 52. The Petri dish sample holder assembly 55 comprises a Petri dish 54, a Petri dish sample holder base 41, a white LED backlight 46, and a backlight base 47. As noted supra, the sample base 41, and backlight base 47 are 3D printable.

As best shown in FIG. 3, the system 30 may alternatively comprise a well plate-type sample holder/XY moving stage 70 for analyzing collected samples that are cultivated in various possible selected well plates 75. For the purposes of this disclosure, a “selected well plate” 75 refers to any one of the well plates 72, 74, 76, 78 shown in FIG. 3, or other similarly sized well plates having at least one “well” area recessed into a flat surface of the plates. In the preferred embodiment, the well/dip/dent/impression/dimple/indent is geometrically shaped, however, in alternative embodiments the well may have any shape in the art consistent with holding a sample.

Significantly, the XY moving stage 70 for holding a selected well plate 75 is functionally identical to the XY moving stage 52 for holding a Petri dish assembly 55.

In addition to an exemplary well plate-based moving stage 70, FIG. 3 shows multiple specific selected well plate 75 designs 72, 74, 76, 78. The selected well plates 75 shown in FIG. 3 were custom designed and manufactured to hold solid or liquid samples/sample spots with different sizes or volumes, and they were made of hard aluminum with nickel surfaces to minimize Raman and fluorescence signals from the sample holder. The newly created modular selected well plates 75 shown in FIG. 3 are more flexible and convenient for sampling than standard plastic and glass microplates (e.g., 96-well plate) used in commercial spectroscopy systems.

When sampling a selected well plate 75, the initial position of the plate on the XY stage is predetermined to align the laser beam probe 37, 39 with the first well holding the sample (usually at top left corner of the plate). Along both X and Y directions of the selected well plate 75, the number of the wells to be sampled and the distance between adjacent wells can be adjusted using the system software, which provides a flexible way to measure the samples placed in selected well plates 75 with different number and size of the wells. As a selected well plate 75 is moved by the XY stage 70, the sample in each well is exposed to a laser component 36, 38 and associated probe 37, 39. Automated sample analysis is then executed in a well-by-well manner. For each sample in the Petri dish 54 or the selected well plate 75, the number of Raman spectra to be collected (e.g., 2×2 or 100×100) and the step size used for XY point scan of each spot (e.g., 0.1 mm or 1.0 mm) can be controlled by the system software to satisfy measurement requirements for different types of the samples. The automated Raman spectral acquisition method for the Petri dish XY moving stage 52 and the selected well plate XY moving stage 70 can be adapted for other point spectroscopy techniques for rapid and flexible sampling, such as reflectance and fluorescence.

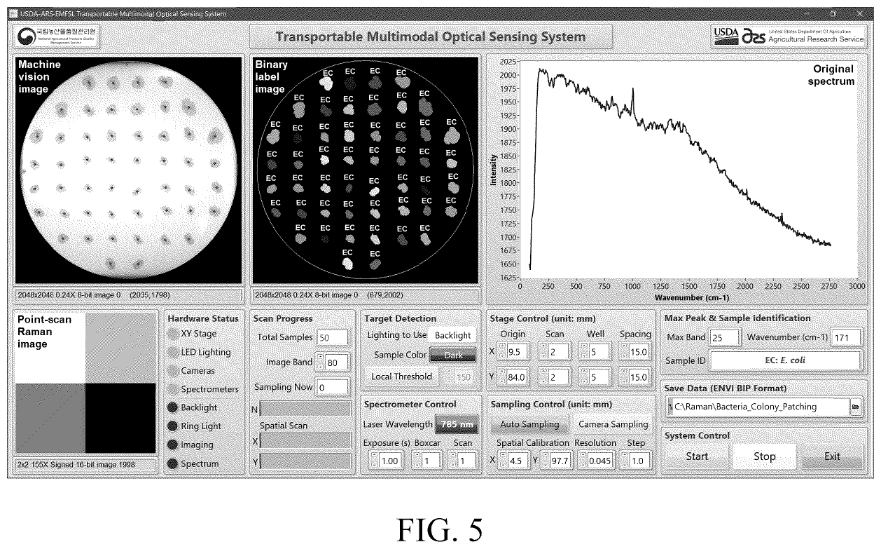

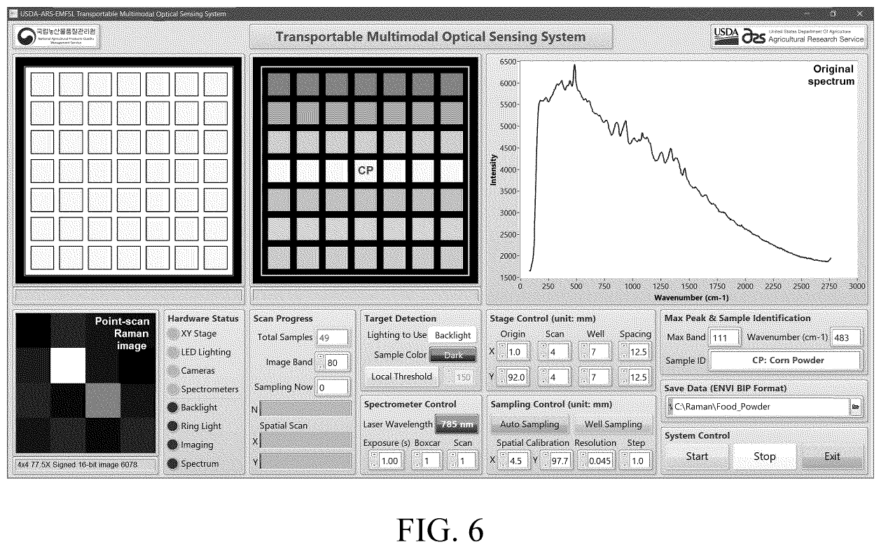

In operation, a computer-based controller/processor exchanges operating instructions and data with the spectrometer units, imaging cameras, lighting hardware, and the XY moving stage and other instruments in the portable multimodal platform via a wired or wireless communication means. As shown in FIG. 1, in the preferred embodiment, the controller/processor 90 comprises a laptop computer that is separate from, but in electronic communication with, the portable multimodal platform instruments. The specific exchange of information and instructions is accomplished via electrical technology that is well-known in the computer and mechanical arts. FIGS. 5 and 6 show examples of system interface screens whereby the computer/processor communicates with the instruments in the portable multimodal platform.

### Flow Chart

A flow chart of the current sample analysis process is shown in FIG. 4. An operator places a Petri dish 54, a selected well plate 75, or other sample holder on an XY moving stage 70, 52. The operator then keys in at least the preferred “Scan Number” and a step size for X and Y directions into the controller/processor for each identified sampling spot in a selected sample holder 54, 75. A “Scan Number” is a parameter for scanning each spot in the Petri dish (or well plate). The scan number is entered in the “Stage Control” sections of the control screen shown in FIGS. 5 and 6. The scan number is predetermined for auto sampling for both the Petri dish and well plate embodiments.

The operator also keys in information regarding whether the sample/analyte is in a Petri dish 54 or a selected well plate 75. If the analyte is in a Petri dish 54, the Petri dish (including the analyte) is moved into a field of view of a color camera (58 or 60). Once the Petri dish 54 is in position, the analyte is illuminated by an LED light, (e.g., a white backlight 46, UV ring light 48, or a white ring light 50) and an image is acquired by the relevant camera 58, 60.

The image is processed in real time to determine the number and position of all the sample spots in the Petri dish 54. The Petri dish 54 is then moved to align a sample spot with either the 785 nm laser probe 37, or the 1064 nm laser probe 39. Spectra are then acquired for each identified sample spot using the previously selected scan number and step size for the X and Y directions. The acquired data and (optionally) the real time identification results are shown in the Petri dish imaging software—as shown in FIG. 5.

The system is then queried regarding whether the currently analyzed sample spot is the last sample spot in the Petri dish 54. If other unanalyzed sample spots remain, then the Petri dish 54 is repositioned to align the 785 nm laser probe 37, or the 1064 nm laser probe 39 with one of the remaining unanalyzed sample spots—and the process described supra repeats itself—until all the sample spots have been analyzed.

After all sample spots are analyzed, the Petri dish 54 is returned to its original position and the acquired data is processed and presented in a format that is usable by the operator. FIG. 5 shows an embodiment of a visual display of all the data associated with the Petri dish 54.

With regard to the well plate 75 sample analysis process, an operator initially places a selected well plate 75 containing analyte samples onto an XY moving stage 70. The operator then enters/inputs a scan number and XY step size into the system analysis software. The operator also enters/inputs the number of wells on the well plate 75 and the distance between adjacent wells. The well plate XY moving stage 70 then moves the selected well plate 75 so that a designated first well aligns with either the 785 nm laser probe 37, or the 1064 nm laser probe 39. Spectra are then acquired for each identified well using the previously selected scan number and step size for the X and Y directions. The acquired data and the real time identification results are shown in the well plate sampling software—as shown in FIG. 6.

The system is then queried regarding whether the currently analyzed sample well is the last well on the well plate 75. If other unanalyzed sample wells remain, then the well plate 75 is repositioned to align the 785 nm laser probe 37, or the 1064 nm laser probe 37 with one of the remaining unanalyzed sample wells—and the process described supra repeats itself—until all the wells in the well plate have been analyzed.

After all the wells are analyzed, the well plate 75 is returned to its original position and the acquired data is processed and presented in a format that is usable by the operator. FIG. 6 shows an embodiment of a visual display of an acquired spectrum and identification result (in “Sample ID”) for one well in a 7×7 well plate.

### System Software

Software for the multimodal sensing system was developed using Lab VIEW (v2017, National Instruments, Austin, Tex.) in Microsoft Windows operating system on a laptop computer. An exemplary color display format associated with the Petri dish embodiment is shown in FIG. 5. An exemplary color display format associated with the well plate embodiment is shown in FIG. 6. The communications between the computer 90 and major hardware components in the system 30, including XY moving stage 52, 70, LED lights 46, 48, 50, machine vision cameras 58, 60, and Raman spectrometers 42, 44, are established using software development kits (SDKs) provided by the hardware manufacturers. The parameterization and data-transfer tasks were realized using functions from both SDKs and LabVIEW, such as User Datagram Protocol (UDP) for LED light control, Vision Development Module (VDM) for camera control and image processing, and Universal Serial Bus (USB) for spectral acquisition and stage movement and synchronization. The manual and automated spectral sampling methods can be controlled by the software. For manual measurement of a single sample, Raman spectral image data are saved in a standard format of band interleaved by pixel (BIP). For automated measurement of multiple samples in a Petri dish 54 or a selected well plate 75, the data are saved in a series of BIP files with sequential numbers automatically appended to the end of the filename (i.e., filename_1, filename_2, . . . , filename_n, in which n is the total number of the samples). During data collection, an original Raman spectrum and a single-band point-scan image are displayed and updated point by point to show the spectral and spatial scan progress in real time. When sampling a Petri dish 54, the software will show a machine vision image and a binary label image of the dish, by which the samples and their center positions (marked by red dots) can be viewed and the sampling process can be monitored.

The saved data can be analyzed offline using commercial software packages such as ENVI (Harris Geospatial Solutions, Broomfield, Colo.) or in-house developed programs by MATLAB (MathWorks, Natick, Mass.). Meanwhile, real-time image and spectral processing and artificial intelligence functions are integrated into the system software, which can be used to identify and label interesting targets in samples using pre-established classification models.

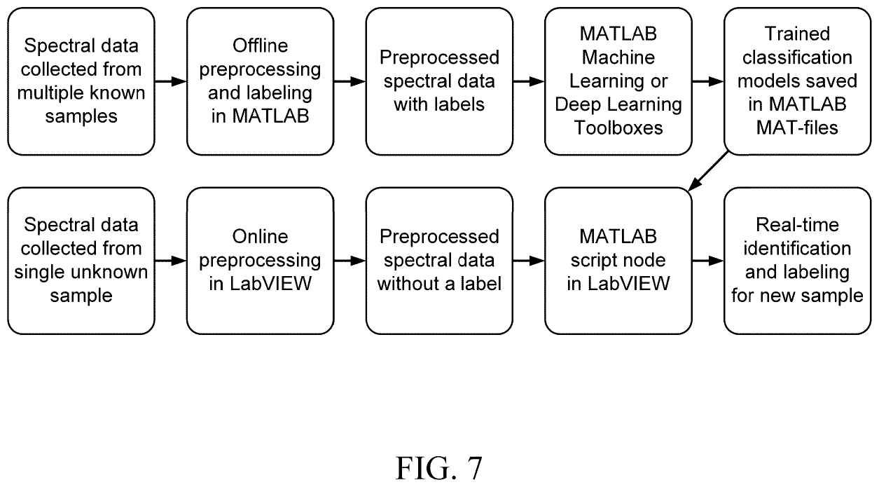

FIG. 7 shows a flowchart of implementing embedded AI functions using MATLAB for offline training and Lab VIEW for online predicting. Spectral data collected from multiple samples in different known classes are first preprocessed and labeled in MATLAB. Then the preprocessed labeled data are input to MATLAB Statistics and Machine Learning or Deep Learning Toolboxes to develop the classification models, which can be exported and saved in standard MAT-files. When measuring a new sample, the spectral data is acquired and preprocessed in LabVIEW using same procedures in MATLAB. The preprocessed unlabeled data and the pre-saved classification model are fed into a MATLAB script node in Lab VIEW, which uses ActiveX technique to execute a program written in MATLAB language syntax to make an instant prediction for the class of the unknown sample. Finally, the class label is shown in the software interface (“Sample ID” in FIGS. 5 and 6) and also appeared above the corresponding sample in the binary image using the center position of the spot. Classification models created using software other than MATLAB, such as TensorFlow and PyTorch, can also be integrated into the Lab VIEW system software using similar manner.

### System Calibration

Spectral calibrations for 785 and 1064 nm Raman spectrometers were conducted using two granular chemicals (polystyrene and naphthalene) as Raman shift standards. When excited by a laser with a fixed wavelength, the chemical standards can generate spectral peaks with known relative Raman shift positions (i.e., wavenumbers), which can be used to calibrate the Raman spectroscopy and imaging systems. For each of the two chemical standards, a pure sample was placed in a Petri dish with a diameter of 47 mm. For each chemical, a 4×4 point scan with a step size of 1 mm for both X and Y directions was performed on the Petri dish sample using both 785 and 1064 nm laser-spectrometer combinations.

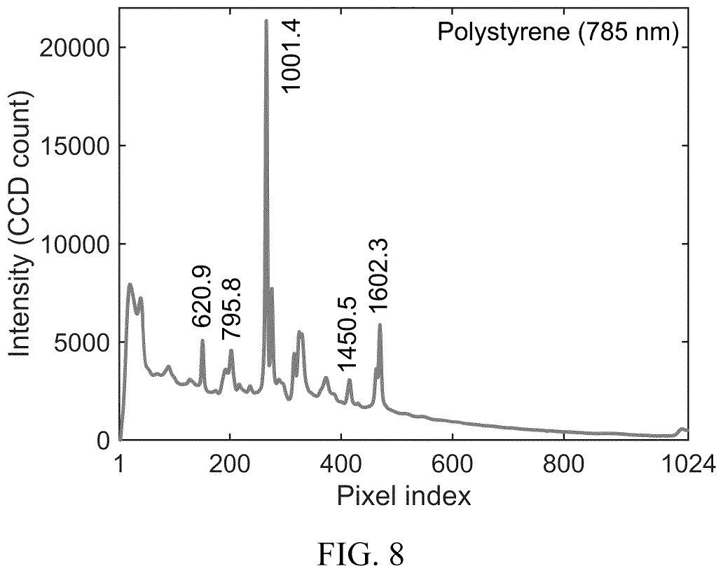

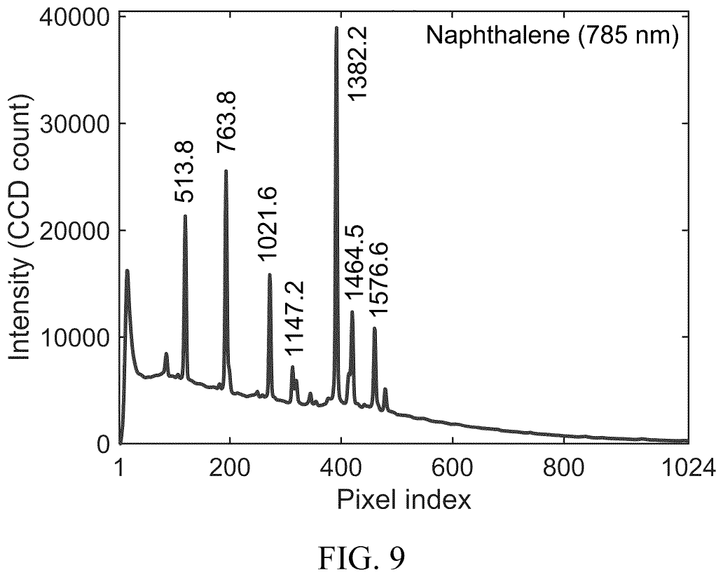

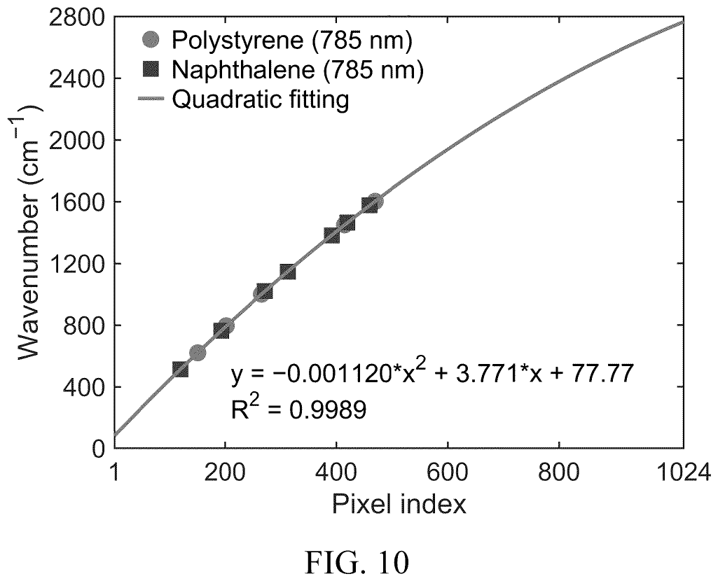

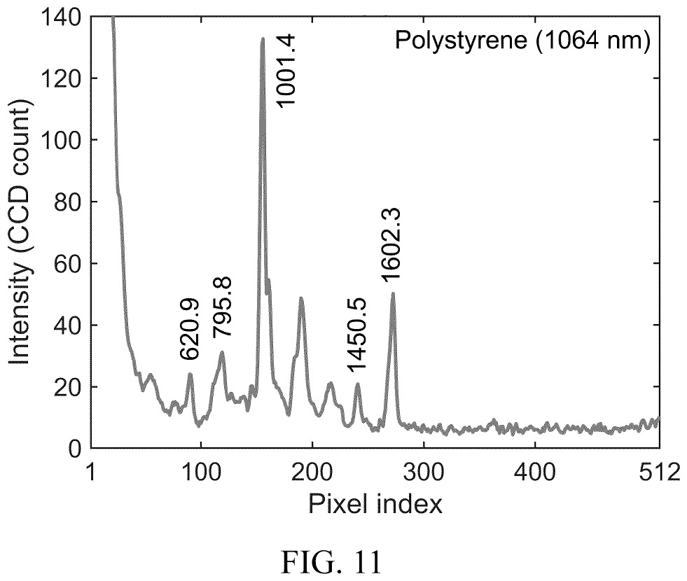

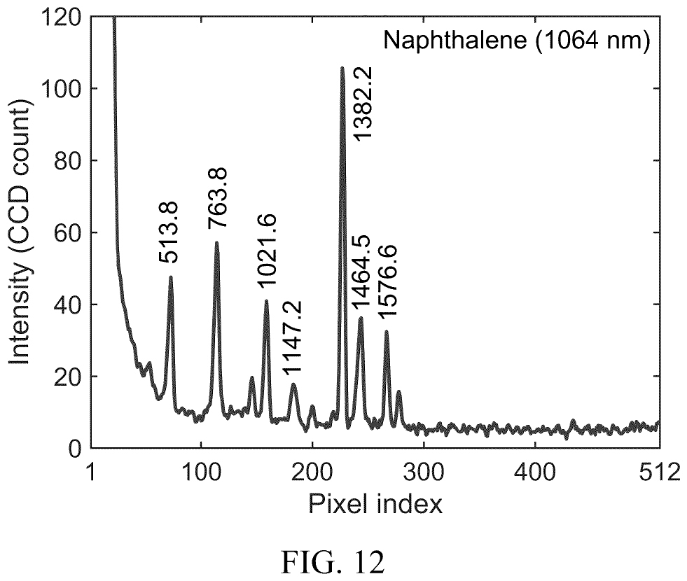

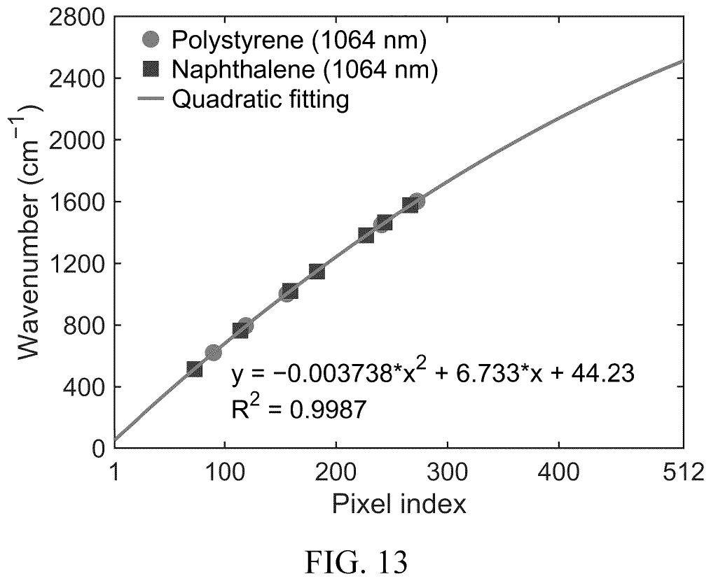

FIGS. 8-13 shows the spectral calibration results. Mean Raman spectra of the two chemical standards were obtained by averaging 16 spectra extracted from the scan area of 4×4 pixels, and the results were plotted in FIGS. 8-9 for 785 nm and FIGS. 11-12 for 1064 nm. A total of 12 Raman peaks (five from polystyrene and seven from naphthalene) were selected to establish the relationship between the wavenumbers and the pixel indices. Regression analyses were conducted using quadratic functions for the 785 nm and 1064 nm laser-spectrometer wavelengths. As best shown in FIG. 10, the models yielded an R² of 0.9989 for 785 nm. As best shown in FIG. 13, the models yielded 0.9987 for 1064 nm. Based on the two quadratic regression models, the Raman shift ranges of the system are 81.5-2765.2 cm⁻¹ with a mean wavenumber interval of 2.6 cm⁻¹ for the 785 nm laser-spectrometer and 51.0-2511.8 cm⁻¹ with a mean wavenumber interval of 4.8 cm⁻¹ for the 1064 nm laser-spectrometer.

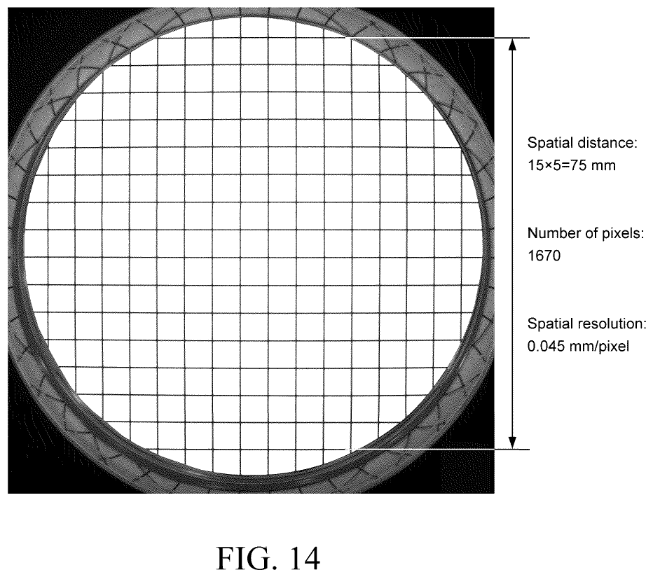

Spatial calibration was carried out for the machine vision cameras to translate the pixel unit in acquired images into the real-world length unit that can be used to drive the XY moving stage 52. Using a working distance (from lens to sample surface) of 156 mm and illumination of the UV ring light 48, an image was collected from a piece of white paper printed with 5 mm square grid that was placed in a 90 mm Petri dish. The original image of 2448×2048 pixels was cropped to 2048×2048 pixels to reduce the unnecessary margin area. The resulting grid is shown in FIG. 14. Under these settings, the field of view of the two machine vision cameras 58, 60 is 92×92 mm² with a spatial resolution of 0.045 mm/pixel. Since the cameras 58, 60 are perpendicular to the plane of the sample surface and no obvious distortion was observed in the image of the grid paper, a simple spatial calibration procedure was used to directly convert the image pixel coordinates to the physical position of the moving stage 52 using the following equations:

$$Dx = Px \times Rx - Cx \qquad ( 1 )$$

$${D}_{Y} = {C}_{Y} - {P}_{Y} \times {R}_{Y} \qquad ( 2 )$$

where D is the physical position in mm, P is the image coordinate in pixels, R is the spatial resolution in mm/pixel, C is the calibration distance in mm, and subscripts X and Y denote the X and Y directions of the image and the moving stage 52. The calibration distances (Cx and Cv) were determined by aligning the 785 nm laser point with round black dots (4 mm diameter and 10 mm center-to-center distance) on a piece of calibration grid paper. Under the current setup, Cx and Cy were determined at 4.5 and 97.7 mm, respectively (shown under “Spatial Calibration” in the system software in FIGS. 5 and 6). The values of Cx and Cy were adjusted for the 1064 nm laser probe 39 to compensate for the offset distance between the two probes 37, 39. For automated Raman measurement of samples randomly scattered in a Petri dish 54, an array of the physical positions (D_{X} and D_{Y}) of the moving stage are calculated by equations 1 and 2 using centers of mass (P_{X} and P_{Y}) of all the sample spots isolated in the binary image.

### Using System to Identify Foodborne Bacteria

The developed system 30 is intended to be used as a general tool for biological and chemical food safety inspection in regulatory and industrial applications. One primary application is rapid, automated, and intelligent identification of foodborne pathogens for regulatory purposes. The capability of the system 30 was demonstrated by an application for identifying common foodborne bacteria prepared using culture-based method.

### Counting and Locating Bacterial Colonies

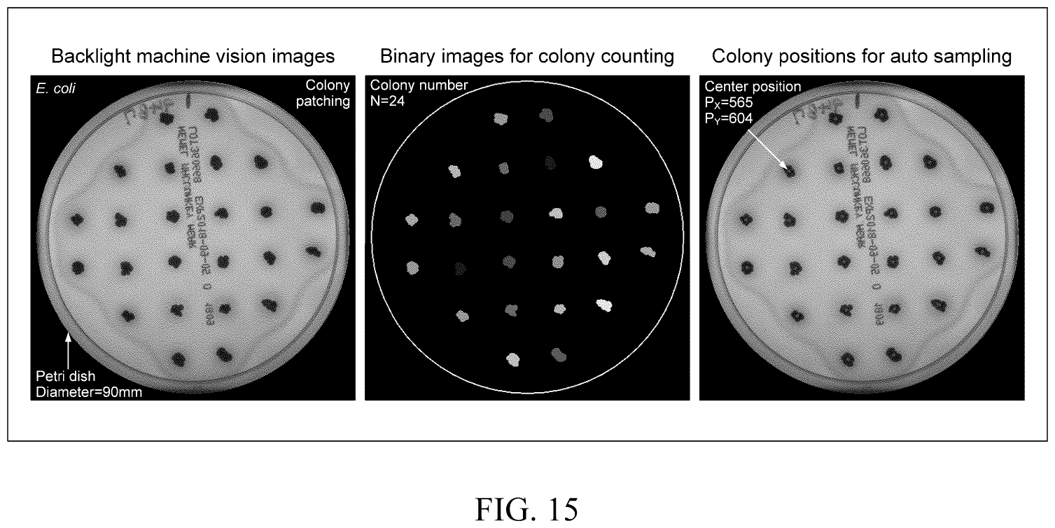

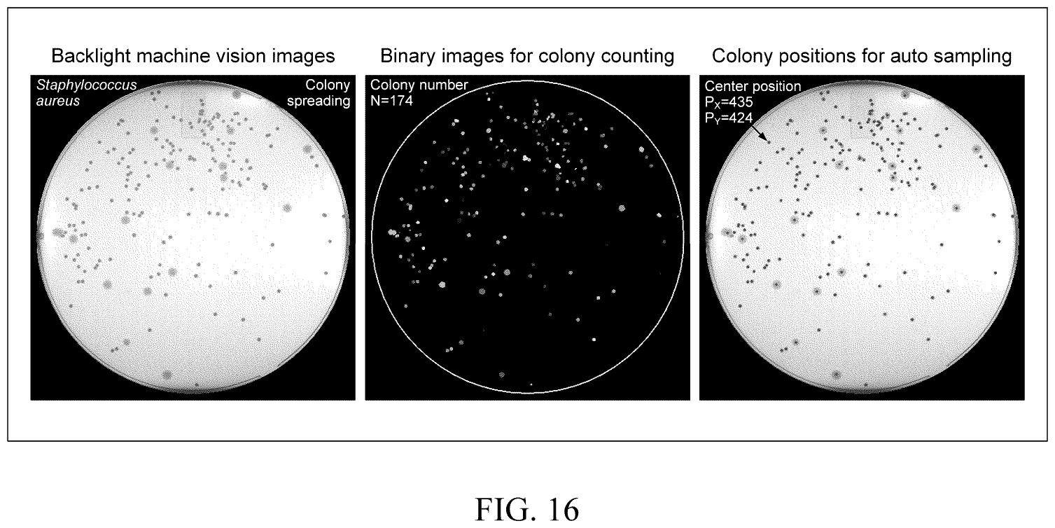

FIGS. 15 and 16 show an example of determining the number and locations of bacterial colonies in Petri dishes for automated Raman spectral measurement. In FIG. 15, E. coli colonies were patched onto a ready-to-use commercial agar plate. In FIG. 16, Staphylococcus aureus colonies were spread on a lab-prepared agar plate. The FIGS. 15 and 16 are used to illustrate the image processing results for the colonies of different sizes with different agar backgrounds. With illumination from the white LED backlight 46, the colonies in the two agar plates can be clearly observed in the grayscale images converted from the original color images.

The grayscale images were then converted to the binary images using a local adaptive thresholding method. Specifically, the “Background Correction” option in Lab VIEW's IMAQ Local Threshold function was used to eliminate non-uniform (FIG. 15) and uniform (FIG. 16) agar backgrounds. The printed and handwritten texts on the bottom of the commercial agar plate (FIG. 15) were removed using the “Heywood Circularity Factor” option in Lab VIEW's IMAQ Particle Filter function. Considering that most bacterial colonies are disk shaped, a circularity range of 0.5-1.5 (the circularity of a circle is 1) was used to exclude non-disk-shaped pixel areas. As a result, only the bacterial colonies were shown in the final binary images. Based on the binary images, the total number and the pixel coordinates of all the colonies were obtained using Lab VIEW's IMAQ Particle Analysis function. Small red dots were overlaid onto the backlight images to mark the centers of mass for all the colonies.

For example, the pixel coordinates (P_{X} and P_{Y}) for the mass center of one E. coli colony in the upper left of the Petri dish (FIG. 15) was determined to be P_{X}=565 and P_{Y}=604. Using equations 1 and 2 (described supra), the physical position of the moving stage can be calculated as D_{X}=565×0.045−4.5 mm=20.92 mm and D_{Y}=97.7−604×0.045 mm=70.52 mm, which can be used to drive this colony to the 785 nm laser beam for the spectral acquisition.

### Bacterial Colony Identification

To explore the potential of the system 30 for identification of foodborne bacteria, bacteria of five common species were prepared for a demonstration experiment. The species included Bacillus cereus from American Type Culture Collection (Manassas, Va.). The species also included E. coli, Listeria monocytogenes, Staphylococcus aureus, and Salmonella spp. from the culture collection in the USDA/ARS Environmental Microbial and Food Safety Laboratory. Isolates from the five bacterial species were grown on same nutrient nonselective agar (BBL, BD, Franklin Lakes, N.J.) in 90 mm Petri dishes.

For each species, one patched agar plate was used for automated spectral measurement from all the individual colonies in the Petri dish. Using the 785 nm laser and an exposure time of 1.0 s for the 785 nm spectrometer, a 2×2 point scan with a step size of 1.0 mm was performed for each colony, and a mean spectrum was calculated and then normalized at the maximum intensity to minimize the effect of background intensity fluctuation. A total of 222 colonies from five Petri dishes were sampled, and their normalized spectra were used for classification.

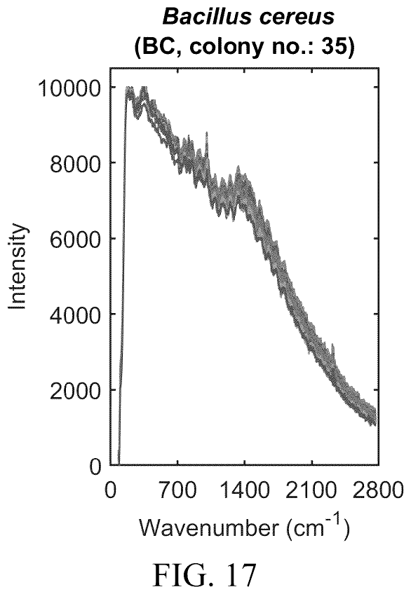

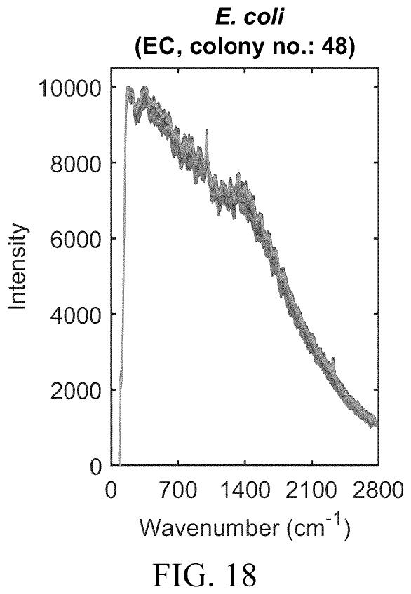

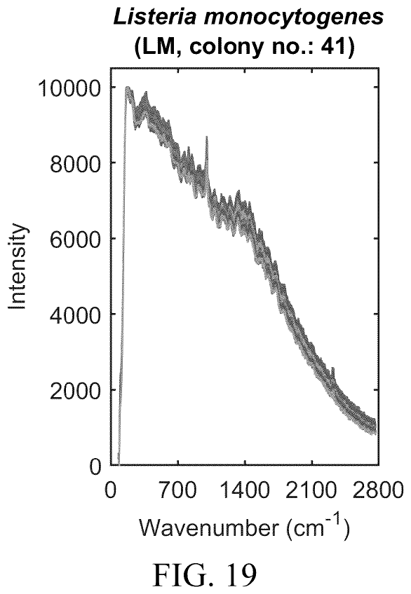

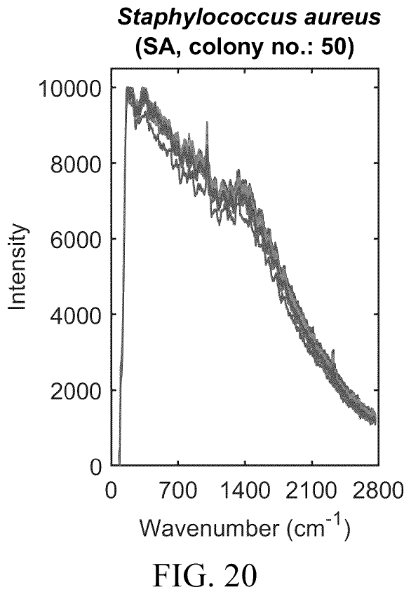

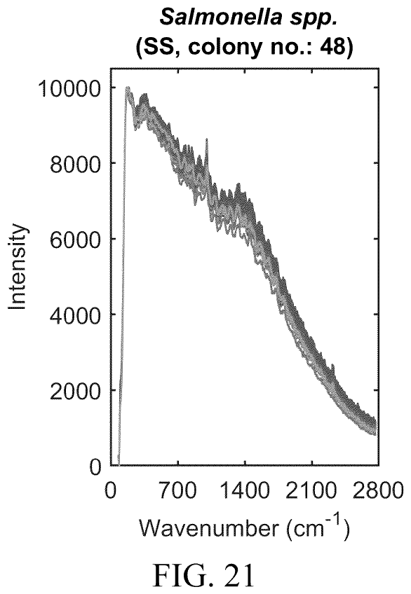

FIGS. 17-21 show the normalized Raman spectra for each of the five bacteria species Specifically FIG. 17 shows Bacillus cereus; FIG. 18 shows E. coli; FIG. 19 shows Listeria monocytogenes; FIG. 20 shows Staphylococcus aureus; and FIG. 21 shows Salmonella spp. Each species is shown with colony numbers in a range of 35-50.

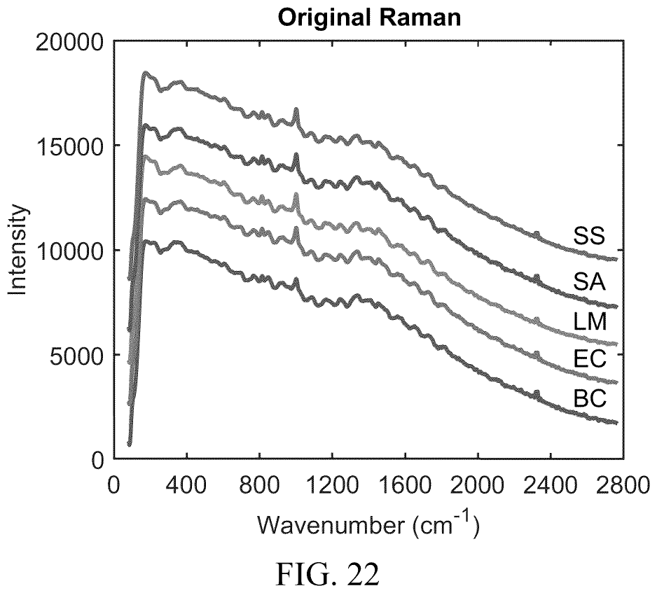

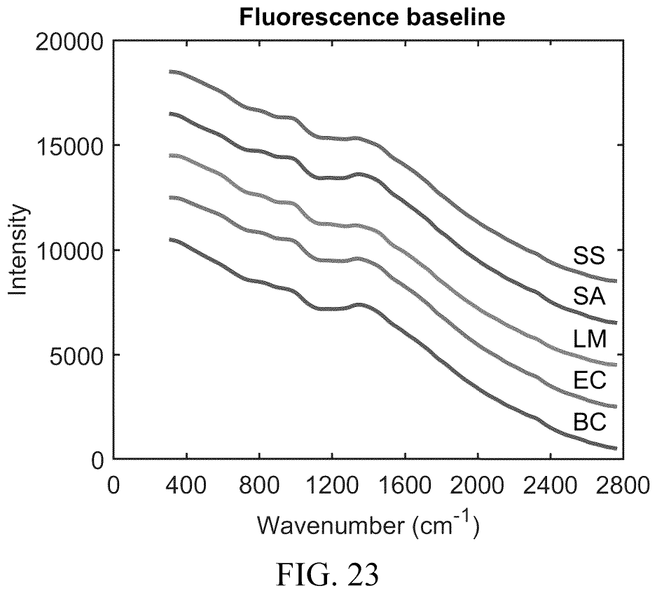

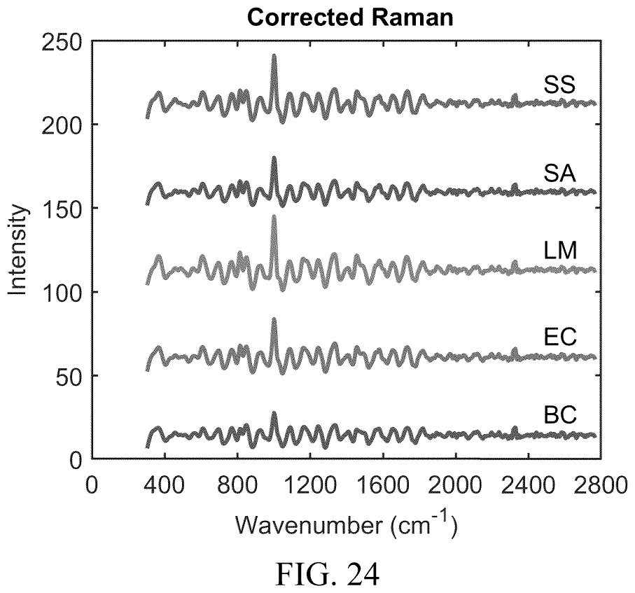

In addition, fluorescence baseline and corrected Raman spectra were obtained from the individual normalized spectra by a baseline correction method using adaptive iteratively reweighted penalized least squares, which were also used for the classifications with the purpose of comparing the performance with the original Raman spectra. The mean spectra of original Raman, fluorescence baseline, and corrected Raman for each species are plotted in FIGS. 22-24. No notable spectral differences were observed from the three types of the data. Machine learning classifications were used to differentiate the five bacterial species.

Note that in FIGS. 22-24: Bacillus cereus is labeled “BC”; E. coli is labeled “EC”; Listeria monocytogenes is labeled “LM”; Staphylococcus aureus is labeled “SA”; and Salmonella spp. is labeled “SS”.

The three types of the spectral data were labeled with the five bacterial species. Each labeled dataset was input to the Classification Learner app in MATLAB (R2021b, MathWorks, Natick, Mass.), in which seven optimizable classifiers, including Naive Bayes, decision tree, ensemble, k-nearest neighbor (KNN), discriminant analysis, neural network (NN), and support vector machine (SVM), were used for the machine learning classifications. Values of hyperparameters for all the models (e.g., model and algorithm parameters such as maximum number of splits for a decision tree, distance metric of a KNN, and box constraint level of an SVM) were automatically selected using the hyperparameter optimization function within the app with a goal of minimizing the classification error. Equal penalty was assigned to all misclassifications to simplify the evaluation of misclassification costs and model training and validation. Five-fold cross-validation method was used to evaluate accuracies of the seven classification models using the three spectral datasets. Each dataset including 222 spectra was randomly partitioned into five disjoint folds. A model was trained using out-of-fold data and the performance was evaluated using in-fold data. The average accuracy was calculated over all folds. To minimize variations from random dataset partitioning, training and validation of each model was repeated ten times. The overall accuracy of each classification model was obtained by calculating the average error over the ten runs.

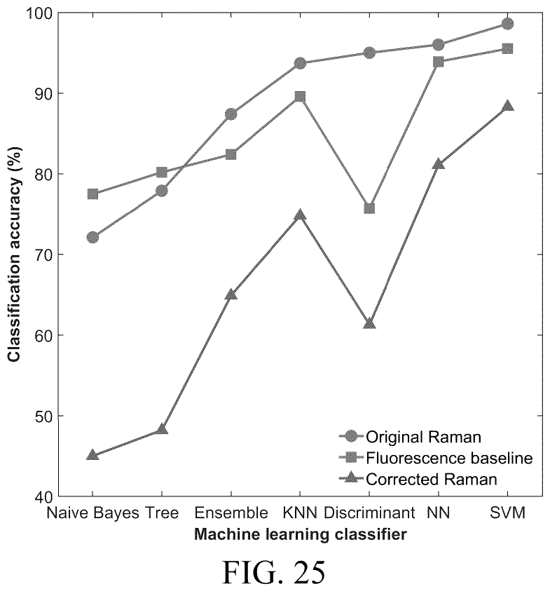

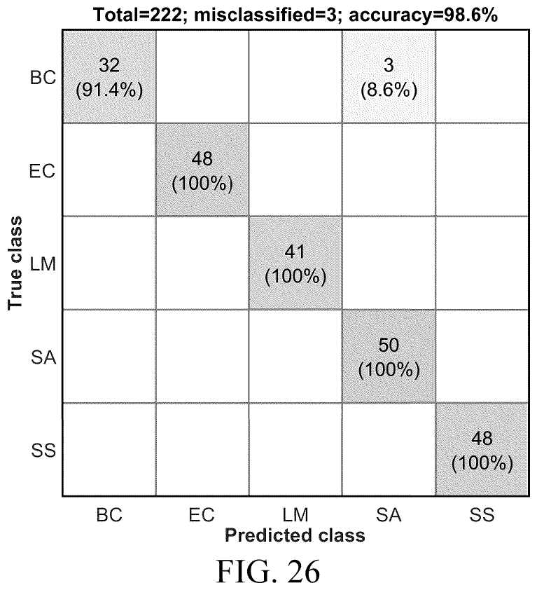

FIG. 25 summarizes accuracies for classifying the five bacterial species using seven optimized models on three spectral datasets. The 21 data points in FIG. 25 reveal that different combinations of the classifier and the dataset resulted in different accuracies. The accuracies of the Naive Bayes and decision tree classifiers using the fluorescence baseline spectra were better than those using the original Raman spectra. Except for these two models, the original Raman dataset outperformed the fluorescence baseline dataset for the other five classifiers, and the corrected Raman dataset yielded the lowest accuracies for all the seven classifiers. Also, the performance of the SVM classifiers was better than the other six classifiers. The highest accuracy was obtained using the optimized SVM model with a linear kernel function on the original Raman spectra, and its confusion matrix is shown in FIG. 26.

Four bacterial species, including E. coli, Listeria monocytogenes, Staphylococcus aureus, and Salmonella spp., were perfectly classified. Only three Bacillus cereus colonies (out of a total of 222 samples) were misclassified as Staphylococcus aureus, and the overall accuracy was achieved at 98.6%. These results suggested that both Raman and fluorescence signals from the colonies grown on the agar contributed to the bacterial species differentiation, and machine learning classification models compensated for small spectral differences among the five species. The trained classification models can be saved and used in the Lab VIEW system software to make real-time predictions for newly collected spectral data, which is faster than the existing detection techniques, such as polymerase chain reaction (PCR) and enzyme-linked immunosorbent assay (ELISA). This demonstration example was carried out using a limited number of bacterial species and colonies on a single agar background. The system is also capable of more complex work directed to more species, colonies, and agars for rapid, automated, and intelligent identification of foodborne bacteria.

### CONCLUSION

The invention described herein comprises an innovative multimodal optical sensing system and protocol based on dual-band laser Raman spectroscopy. The system hardware, software, and system integration techniques provide a new tool for automated and intelligent food safety inspection. Two pairs of lasers and spectrometers working at different wavelengths enable high-quality Raman scattering signals to be obtained from both low- and high-fluorescence food samples in a single measurement system. By utilizing machine vision and motion control techniques, the system can conduct fully automated spectral acquisition for samples randomly scattered in Petri dishes or placed in customized well plates, which is more flexible and versatile than commercial integrated Raman systems using standard microplates. The concept of automated spectral collection can be extended beyond Raman to other spectroscopy techniques.

Interesting targets in the samples can be identified and labeled using real-time image and spectral processing and artificial intelligence functions that are integrated into the in-house developed system software. Classification models that use machine learning approaches can compensate for small spectral differences in direct Raman measurements and improve prediction accuracies for various food safety applications. The system is intended to be used by food safety regulatory agencies as an initial screening tool for quick species identification of common foodborne bacteria. The prototype is compact and easily portable, which makes it suitable for field and on-site food safety inspections in wide variety of industrial and regulatory applications.

For the foregoing reasons, it is clear that the subject matter described herein provides an innovative portable multimodal optical sensing system that may be used in multiple varying applications. The current system may be modified in multiple ways and applied in various technological applications. The disclosed method and apparatus may be modified and customized as required by a specific operation or application, and the individual components may be modified and defined, as required, to achieve the desired result.

Although most of the materials of construction are not described, they may include a variety of compositions consistent with the function described herein. Such variations are not to be regarded as a departure from the spirit and scope of this disclosure, and all such modifications as would be obvious to one skilled in the art are intended to be included within the scope of the following claims.

The amounts, percentages and ranges disclosed in this specification are not meant to be limiting, and increments between the recited amounts, percentages and ranges are specifically envisioned as part of the invention. All ranges and parameters disclosed herein are understood to encompass any and all sub-ranges subsumed therein, and every number between the endpoints. For example, a stated range of “1 to 10” should be considered to include any and all sub-ranges between (and inclusive of) the minimum value of 1 and the maximum value of 10 including all integer values and decimal values; that is, all sub-ranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions, and so forth as used in the specification and claims are to be understood as being modified in all instances by the implied term “about.” If the (stated or implied) term “about” precedes a numerically quantifiable measurement, that measurement is assumed to vary by as much as 10%. Essentially, as used herein, the term “about” refers to a quantity, level, value, or amount that varies by as much 10% to a reference quantity, level, value, or amount. Accordingly, unless otherwise indicated, the numerical properties set forth in the following specification and claims are approximations that may vary depending on the desired properties sought to be obtained in embodiments of the present invention.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as 10% to a reference quantity, level, value, or amount.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein). The invention illustratively disclosed herein suitably may be practiced in the absence of any element which is not specifically disclosed herein. The term “an effective amount” as applied to a component or a function excludes trace amounts of the component, or the presence of a component or a function in a form or a way that one of ordinary skill would consider not to have a material effect on an associated product or process.

What is claimed is:

1. A portable multimodal optical sensing system, the system comprising:
a sample holder connected to an XY moving base, the sample holder holding at least one analyte sample;
at least two cameras;
at least two Raman laser excitation sources;
at least two Raman spectrometers;
at least two illumination sources, the illumination sources comprising at least a UV light and an analyte sample backlight;
a portable base and housing at least partially enclosing the XY moving base, the cameras, the Raman excitation sources, and the illumination sources; and,
a computer/processor in communication with and controlling the XY moving base, the cameras, the Raman excitation sources, and the illumination sources;
wherein the system is structured so that the computer/processor directs the XY moving stage to enable at least one of the cameras to scan the at least one analyte sample so that the computer/processor determines an analysis protocol, the XY moving stage being configured to move in accordance with the analysis protocol so that the at least one analyte sample is analyzed.

2. The system of claim 1 wherein the computer/processor is external to the portable base and housing.

3. The system of claim 1 wherein the at least one analyte sample in the holder comprises a macro-scale analyte sample.

4. The system of claim 1 wherein the sample holder comprises a well plate or a Petri dish.

5. The system of claim 1 wherein each of the at least two cameras has separate imaging acquisition apertures.

6. The system of claim 1 wherein the at least two cameras comprise at least two color cameras.

7. The system of claim 1 wherein at least one of the at least two color cameras comprise a multi-band bandpass filter.

8. The system of claim 1 wherein each of the at least two Raman excitation sources has a separate laser probe;

9. The system of claim 1 wherein at least one of the at least two Raman laser excitation sources comprises a 785 nm laser source for low-fluorescence analyte samples.

10. The system of claim 1 wherein at least one of the at least two Raman laser excitation sources comprises a 1064 nm laser for high-fluorescence analyte samples.

11. The system of claim 1 wherein the UV light comprises a vertically adjustable UV ring light.

12. The system of claim 1 wherein the illumination sources further comprise a vertically adjustable white ring light.

13. The system of claim 1 wherein the computer/processor is structured to use embedded AI to determine the components and the analysis protocol to examine the respective analyte samples.

14. The system of claim 1 wherein if a bacterial species or chemical contaminant is present, the system is structured to identify the bacterial species or chemical contaminant.

15. The system of claim 1 wherein computer/processor is structured to use embedded AI to identify the bacterial species or chemical contaminant in the analyte sample.

16. The system of claim 1 wherein the computer/processor is structured to determine whether a bacterial species or chemical contaminant is present in the at least one analyte sample.

17. The system of claim 15 wherein the computer/processor is structured to identify the bacterial species or chemical contaminant in the analyte sample in real time.

18. A method of analyzing a selected analyte sample, the method comprising:
(a) providing the system of claim 1;
(b) inputting the scan number and step size for X and Y directions for each of the at least one analyte samples into the computer/processor;
(c) moving the sample holder so that the at least one analyte sample is in a field of view of one of the at the least two cameras;
(d) acquiring an image of the at least one analyte sample using an illumination source;
(e) moving the sample holder to align the at least one analyte sample with one of the at least two Raman laser sources and acquiring Raman spectra data for the at least one analyte sample;
(f) saving the at least one analyte sample data acquired in step (e) in an electronic file in the computer/processor;
(g) if the at least one analyte sample is not the last analyte sample in the sample holder, repeating steps (e) and (f) until the last analyte sample in the sample holder is analyzed;
(h) if the at least one analyte sample is the last analyte sample in the sample holder, the computer/processor processing the electronic file data associated with each one of the analyzed at least one analyte sample; and,
(i) determining whether a bacterial species or chemical contaminant is present in each analyzed analyte sample, and if a bacterial species or chemical contaminant is present, identifying the bacterial species or chemical contaminant.

19. The method of claim 17 wherein the sample comprises a macro-level sample.

20. The method of claim 17 wherein, in step (i), the system makes a real time determination regarding whether a bacterial species is present, and if a bacterial species or chemical contaminant is present, then the system identifies the bacterial species or chemical contaminant in real time.

21. The method of claim 17 wherein in step (e), the at least two Raman laser sources comprise at least a 785 nm laser or a 1064 nm laser.

22. The method of claim 17 wherein, in step (i), the system uses embedded AI to identify the bacterial species or chemical contaminant using one type or fusion of multimodal sensing data from Raman spectra, fluorescence images, color images, and transmission images.
